(19) United States
(12) Patent Application Publication
(10) Pub. No.: US 2024/0242528 A1
(43) Pub. Date: Jul. 18, 2024

(54) VERTEBRAL RECOGNITION PROCESS

(71) Applicant: WENZEL SPINE, INC., Austin, TX (US)

(72) Inventor: Seyfettin KULGU, Austin, TX (US)

(73) Assignee: WENZEL SPINE, INC., Austin, TX (US)

(21) Appl. No.: 18/361,961

(22) Filed: Jul. 31, 2023

Related U.S. Application Data
(60) Provisional application No. 63/370,688, filed on Aug. 8, 2022.

(51) Int. Cl. G06V 40/10; G06V 10/764; G06V 10/82; G16H 15/00; G16H 20/40

(52) U.S. Cl. CPC G06V 40/10; G06V 10/764; G06V 10/82; G16H 15/00; G16H 20/40; G06V 2201/033

(57) ABSTRACT

A system and method that includes storing a software application on a memory associated with a computer, which when executed by a processor causes the software application to develop a model of at least a portion of a spine, process images of the spine, recognize the vertebral bodies in the image, map the vertebral bodies, and display the images of the spine on a user interface associated with the computer. The mapped images can be use to develop a prediction model and/or a surgical plan.

### CROSS-REFERENCE

This application claims the benefit of U.S. Provisional Application No. 63/370,688, filed Aug. 8, 2022, entitled VERTEBRAL RECOGNITION PROCESS which application is incorporated herein in its entirety by reference.

### NOTICE OF COPYRIGHTS

A portion of the disclosure of this patent document contains material which is subject to copyright protection. The copyright owner has no objection to the facsimile reproduction of the patent disclosure as it appears in the Patent and Trademark Office patent files or records, but otherwise reserves all copyright rights whatsoever.

### BACKGROUND

A prevalent joint problem is back pain, particularly in the “small of the back” or lumbosacral (L4-S1) region, shown in FIG. 1A. In many cases, the pain severely limits a person's functional ability and quality of life. Such pain can result from a variety of spinal pathologies. Through disease or injury, the vertebral bodies, intervertebral discs, laminae, spinous process, articular processes, or facets of one or more spinal vertebrae can become damaged, such that the vertebrae no longer articulate or properly align with each other. This can result in an undesired anatomy, loss of mobility, and pain or discomfort. Patients suffering from back pain in the United States consume more than $3.1 trillion. Additionally, there is a substantial impact on the productivity of workers as a result of lost work days. As a result of this problem, better and less invasive orthopedic intervention devices and procedures are constantly being developed. Interventions include fixing the spine and/or sacral bone adjacent the vertebra, as well as attaching devices used for fixation. Another intervention is the spinal treatment decompressive laminectomy. Where spinal stenosis (or other spinal pathology) results in a narrowing of the spinal canal and/or the intervertebral foramen (through which the spinal nerves exit the spine), and neural impingement, compression and/or pain results, the tissue(s) (hard and/or soft tissues) causing the narrowing may need to be resected and/or removed.

Evaluating interventions starts with imaging the spine. A variety of imaging methods are available. Spine X-rays are typically taken in either the anteroposterior (front to back) or the posteroanterior (back to front) view, commonly referred to as an AP/PA view, or the lateral (side) view. Computed tomography (CT or CT scan) is a noninvasive diagnostic imaging procedure that uses a combination of X-rays and computer technology to produce axial images, often called slices, of the body. In a CT scan, the X-ray beam moves in a circle around the body. The X-ray information is sent to a computer that interprets the X-ray data and displays it in a two-dimensional (2D) form on a monitor. Digital geometric processing is used to further generate a three-dimensional (3D) volume of the inside of the subject from the series of 2D images taken around a single axis of rotation during the CT scan. As appreciated by those skilled in the art, CT scans are more detailed than standard X-rays. CT produces data that can be manipulated in order to demonstrate various bodily structures based on their ability to absorb the X-ray beam. CT scans of the spine can provide more detailed information about the vertebrae than standard X-rays, thus providing more information related to injuries and/or diseases of the spine

The extent to which a specific treatable joint defect can be identified and optimally treated directly impacts the success of any treatment protocol. A key to diagnostic techniques is to provide measurement data that precisely identifies the vertebral bodies in acquired images.

What is needed are systems and methods for improved vertebral body recognition.

### SUMMARY

Disclosed are systems and methods for improved vertebral body recognition.

Vertebral recognition systems and methods are disclosed. The systems and methods comprise: a computer including a memory, a processor and a display; and an application stored in the memory and executable by the processor of the computer to obtain a plurality of images from one or more databases wherein the plurality of images include a spine having one or more vertebral bodies, map one, two, three, or more vertebral bodies in the one, two, three or more images with at least one of two points and four points to the generated one, two, three or more mapped images of the one, two, three or more of two point vertebra and four point vertebra, create a prediction model using the one or more images, wherein the prediction model is created using the plurality of mapped images, and one or more of a count of epochs and various counts of steps, and build an automated mark-up. The various count of steps is a parameter that represents the number of iterations used to run the algorithm and build a model for an epoch. The step count is dynamic and varies based on the specific model being constructed. It can change depending on the complexity and size of the model under consideration. Similarly, the number of epochs is also dynamic, indicating how many times the algorithm iterates over the entire dataset during the training process. Building a model involves running multiple steps for each epoch, making it a combination of various epochs and their corresponding step counts.

Vertebral recognition systems and methods are also disclosed comprising: a computer including a memory, a processor and a display; and an application stored in the memory and executable by the processor of the computer to obtain a plurality of images from one or more databases wherein the plurality of images include a spine having one or more vertebral bodies, map one or more vertebral bodies in the one or more images with at least one of two points and four points to generated one or more mapped images of one or more two point vertebra and four point vertebra, create a prediction model using the one or more images, wherein the prediction model is created using the plurality of mapped images, and one or more of a count of epochs and various counts of steps, instruct to split the mapped images into a training category and a validation category, and build an automated mark-up.

Methods of performing a surgical procedure are also disclosed. The methods comprise: storing a software application on a memory associated with a computer, which when executed by a processor, causes the processor to obtain a plurality of images from one or more databases wherein the plurality of images include a spine having one or more vertebral bodies, map one or more vertebral bodies in the one or more images with at least one of two points and four points to generated one or more mapped images of one or more of two point vertebra and four point vertebra, create a prediction model using the one or more images, wherein the prediction model is created using the plurality of mapped images, and one or more of a count of epochs and various counts of steps, build an automated mark-up, generate a surgical procedure plan based on the prediction model.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
CN 112102282 A published Dec. 18, 2020 to Qin et al.;
U.S. Pat. No. 5,349,956 A issued Sep. 27, 1994 to Bonutti;
U.S. Pat. No. 5,427,116 A issued Jun. 27, 1995 to Noone;
U.S. Pat. No. 6,560,476 B1 issued May 6, 2003 to Pelletier et al.;
U.S. Pat. No. 7,266,406 B2 issued Sep. 4, 2007 to Kroeckel;
U.S. Pat. No. 7,502,641 B2 issued Mar. 10, 2009 to Breen;
U.S. Pat. No. 8,676,293 B2 issued Mar. 18, 2014 to Breen et al.;
U.S. Pat. No. 10,902,587 B2 issued Jan. 26, 2021 to Manickam et al;.
U.S. Pat. No. 11,145,060 B1 issued Oct. 12, 2021 to Schudlo et al.;
US 2021/0378616 A1 published Dec. 9, 2021 to Chan et al.;
US 2021/0383536 A1 published Dec. 9, 2021 to Schudlo;
US 2022/0083821 A1 published Mar. 17, 2022 to Jiang et al.; WO 2022/090102 A1 published May 5, 2022 to Yaakobe et al.;
KONYA et al., Convolutional neural network based automated segmentation and labeling of the lumbar spine X ray, J. Craniovertebral Junction & Spine 12(2): 136 (2021);
MUSHTAQ, et al., Localization and Edge Based Segmentation of Lumbar Spine Vertebrae to Identify the Deformities Using Deep Learning Models, Sensors 22(4): 1547 (2022);
VANIA et al., Intervertebral disc instance segmentation using a multistage optimization mask RCNN (MOM RCNN), J. Comp. Des. and Engr. 8(4): 1023 1036 (2021); and
WANG, et al., Vertebra Segmentation for Clinical CT Images Using Mask R CNN, EU Med. and Biol, Engr. Conf. pp. 1156 1165 (2020).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a lateral view of a normal human spinal column;

FIG. 1B is illustrates a human body with the planes of the body identified;

FIGS. 2A-C illustrate a subject bending through a range of spinal flexion and extension motion with a corresponding x-ray taken at each position, as currently practiced in the art;

FIG. 2D illustrates stacked vertebral bodies of a spine moving through the range of motion illustrates in FIGS. 2A-C;

FIG. 2E illustrates a process for interpreting radiographic images in traditional spinal kinematic studies;

FIG. 2F is an illustrative inter-vertebral motion curve, corresponding to flexion/extension or side-bending, along with an illustrative representation of error in observed measurement;

FIG. 2G illustrates an actual intervertebral motion curve for the L4-L5 joint of a healthy subject side bending;

FIG. 3 is a block diagram that shows the relationship between an imaging system and a tracking system;

FIGS. 4A-4B illustrate a data extraction process for vertebral recognition;

FIGS. 5A-B illustrate a training process for vertebral recognition;

FIG. 6 illustrates a training process for bend type recognition;

FIG. 7 illustrates a prediction process; and

FIG. 8 illustrates an alternative prediction process.

### DETAILED DESCRIPTION

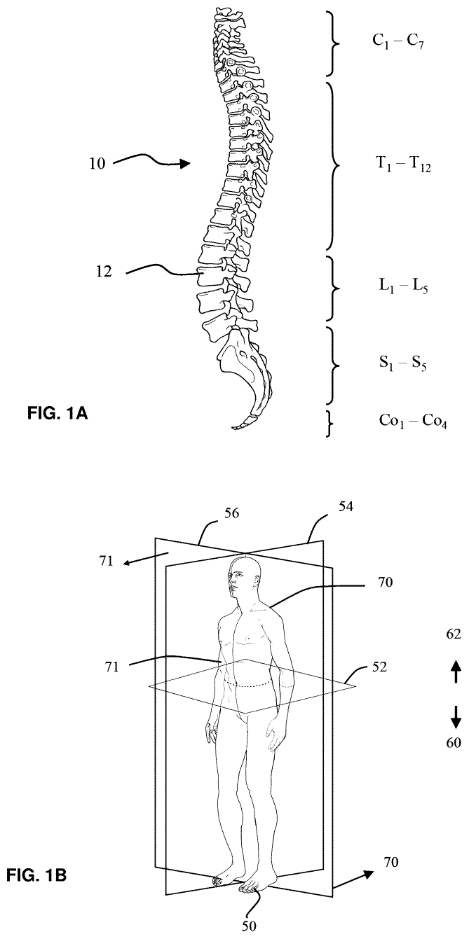

FIG. 1 illustrates the human spinal column 10 which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

As shown in FIG. 1B, the body 50 has are three anatomical planes generally used in anatomy to describe the human body and structure within the human body: the axial plane 52, the sagittal plane 54 and the coronal plane 56. Additionally, devices and the operation of devices and tools may be better understood with respect to the caudad 60 direction and/or the cephalad direction 62. Devices and tools can be positioned dorsally 70 (or posteriorly) such that the placement or operation of the device is toward the back or rear of the body. Alternatively, devices can be positioned ventrally 72 (or anteriorly) such that the placement or operation of the device is toward the front of the body. Various embodiments of the systems and methods of the present disclosure may be configurable and variable with respect to a single anatomical plane or with respect to two or more anatomical planes.

For purposes of illustration, the systems and methods are described below with reference to the spine of the human body as needed.

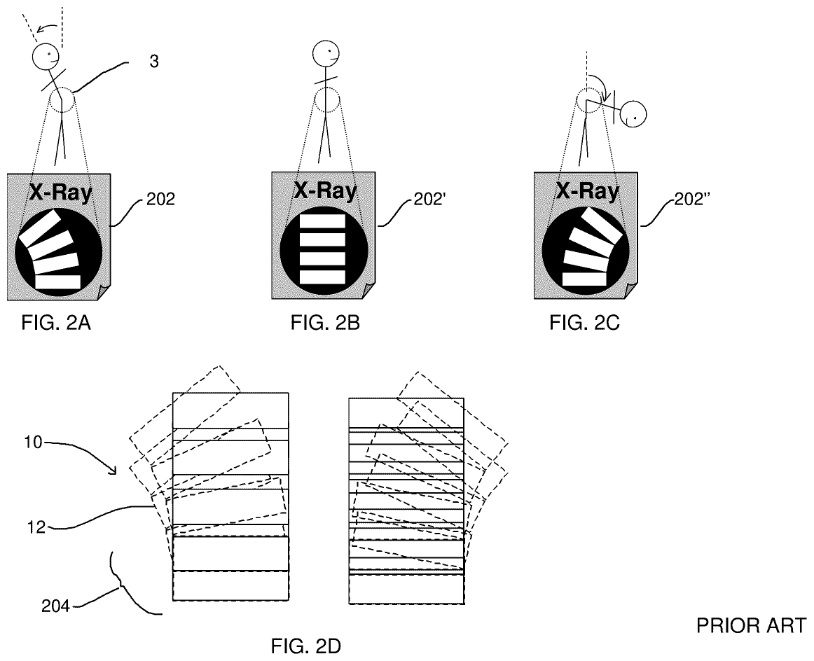

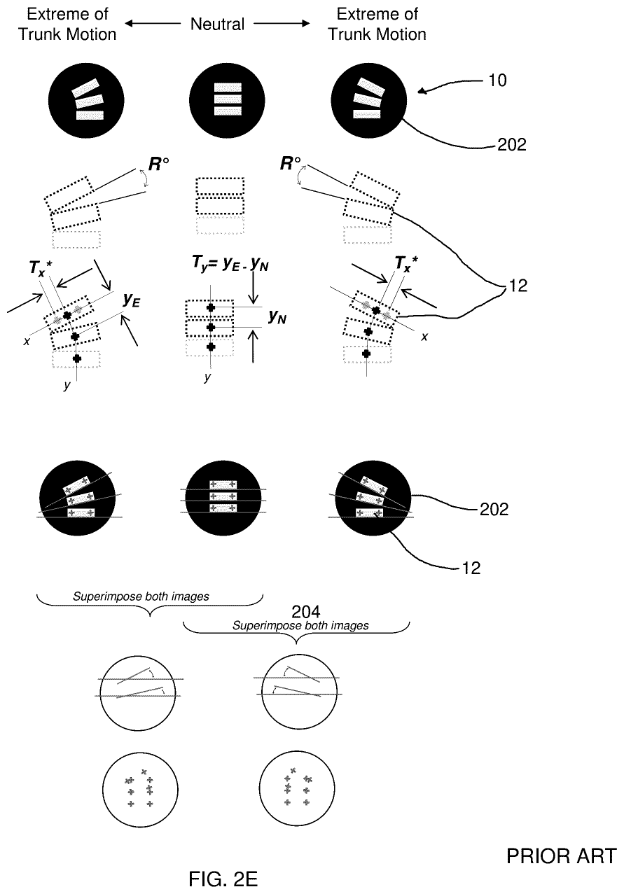

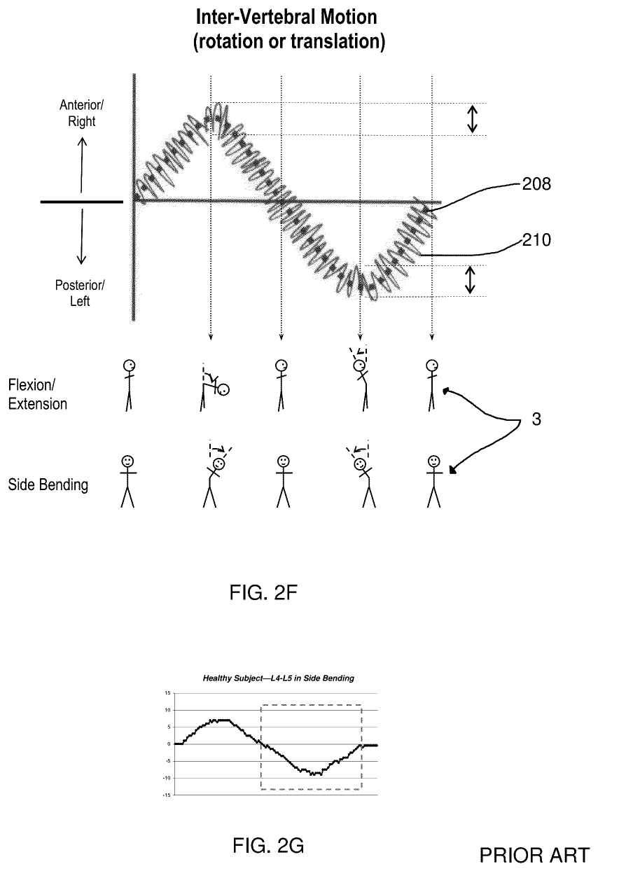

FIGS. 2A-C illustrate a subject 3 bending through a range of motion with a corresponding x-ray 202 taken at each position, as currently practiced in the art. Typically a subject 3 is instructed to stand in front of a device adapted to capture an x-ray image and then bend to a first position and then to a second position. An x-ray image 202, 202′, 202″ is taken at each of these positions. Thereafter two x-rays images, e.g., 202, 202′, are superimposed, e.g. as illustrated in FIG. 2D, to show the vertebral bodies 12 stacked and crudely moving through the range of motion. As illustrated in FIG. 2E this process for interpreting radiographic images in traditional spinal kinematic studies has a variety of manual steps which include using a protractor to draw on the image to measure how much movement has occurred. These steps are also sometimes executed with the assistance of a computer, in which case the manual steps are done with a mouse or other manually-operated computer input device. The results achieved using these manual methods, as shown in FIG. 2F, are inherently subject to a high degree of inter-observer and intra-observer variability as different observers utilize different techniques to landmark the images and derive measurements. Further, the uncontrolled bending process represented in FIGS. 2A-C is responsible for introducing a high degree of inter-subject and intra-subject variability into these measurements as different subjects are capable of bending to differing positions. As will be appreciated by those skilled in the art, any medical image-derived quantitative measurements of joint motion 204 will also contain variability that is due to out of plane and geometric distortions inherent to medical imaging. Therefore, image based measurements for range of motion would exhibit observable measurements that fall within a distribution of variability about the actual motion. Combining these three sources of variability, it is well established that in the clinical utilization of image-derived measurements of intervertebral range of motion, it is not feasible to interpret such measurements as having error of any better than +5°. FIG. 2E shows the mean L1/L2 rotational ROM taken from a normative population of pain free subjects is about 10° of rotation. Accounting for the +5° of error in this measurement, the error bars on this measurement 206 are about 50% of the underlying mean measurement value.

As illustrated in FIG. 2F an inter-vertebral motion curve created taking measurements using currently practiced techniques, corresponding to flexion/extension or side-bending, would have “noise” 212 in the observed motion 210 relative to the actual motion 208. FIG. 2G illustrates an intervertebral motion curve for the L4-L5 joint of a healthy subject side bending.

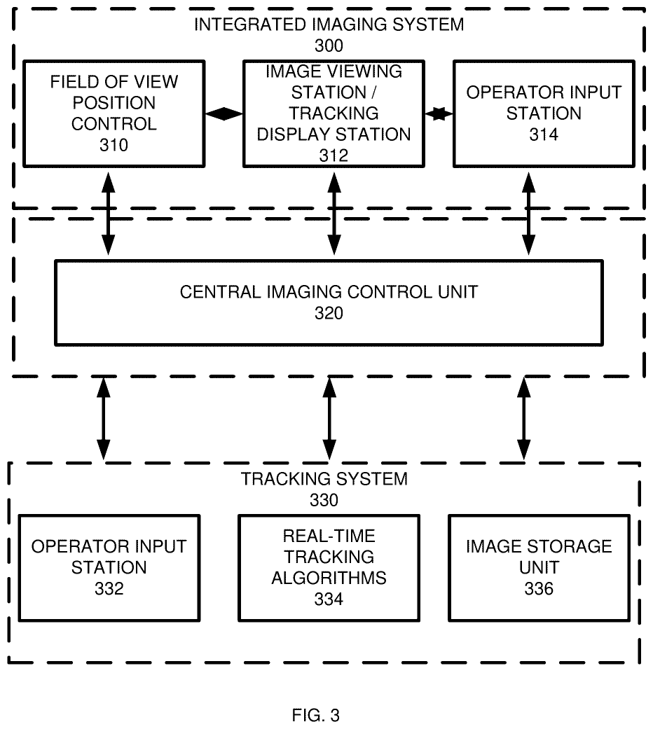

FIG. 3 illustrates a system comprising an integrated imaging system 300 which includes a field of view position control 310, an image viewing stations/tracking display station 312, and an operation input station 314. A central imaging control unit 320 is also provided. The field of view position control 310, image viewing stations/tracking display station 312, operation input station 314, and central imaging control unit 320 are in communication with each of the other components during use. A tracking system 330 is also provided in communication with the integrated imaging system 300. The tracking system 330 further comprises an operator input station 332, real-time tracking algorithms 334 and an image storage unit 336.

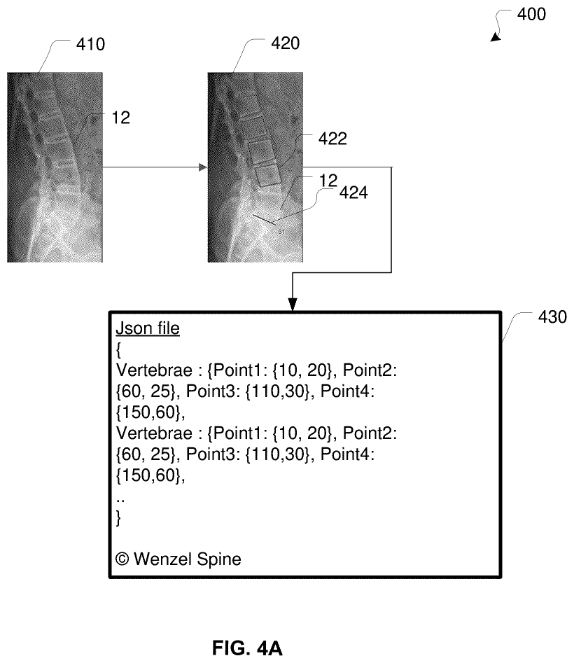

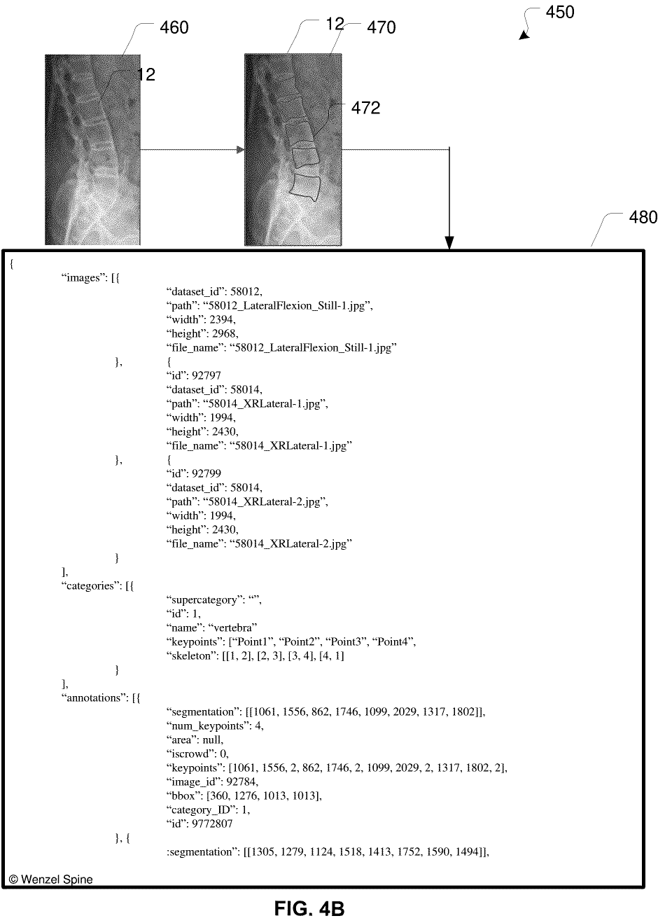

Turning now to FIGS. 4A-B, processes for extracting data for vertebral recognition are shown. FIG. 4A shows a data extraction process 400 wherein a plurality of images, such as x-ray images, are obtained 410. The data extraction process can include gathering images from one or more existing databases. In some configurations the images obtained are jpeg images. The plurality of images can be hundreds, thousands, tens of thousands, etc. A plurality of points 420 are used to locate the vertebral body 12. A plurality of vertebral bodies are mapped with four points 422. For the S1 and C1 vertebral bodies only two points are required for identification, as shown at 424. Information for the two to four points are gathered in a JavaScript Object Notation (JSON) format 430 for training. The plurality of points can be pre-existing on the plurality of images. Additionally, the bend type for the image can also be extracted, e.g., lateral, flexion, extension, anterior-posterior (AP), etc. The data is presented in a single file format, such as JSON, and presented for training via the algorithm.

FIG. 4B shows another data extraction process 450 wherein a plurality of images, such as x-ray images, are obtained 460. The data extraction process can include gathering images from one or more existing databases. In some configurations the images obtained are jpeg images. The plurality of images can be hundreds, thousands, tens of thousands, etc. A plurality of points 470 are used to locate the vertebral body 12. A plurality of vertebral bodies are mapped with four points 472. The images are further annotated to label each of the vertebra (e.g., L1, L2, etc.) while masking them (e.g., annotate the perimeter to essentially create a mask around the perimeter). Information about existing instrumentation between vertebral levels can also be provided, as well as whether there is a fusion. Information is gathered in a large-scale object detection, segmentation, and captioning data set, such as a COCO dataset json format 480 for training.

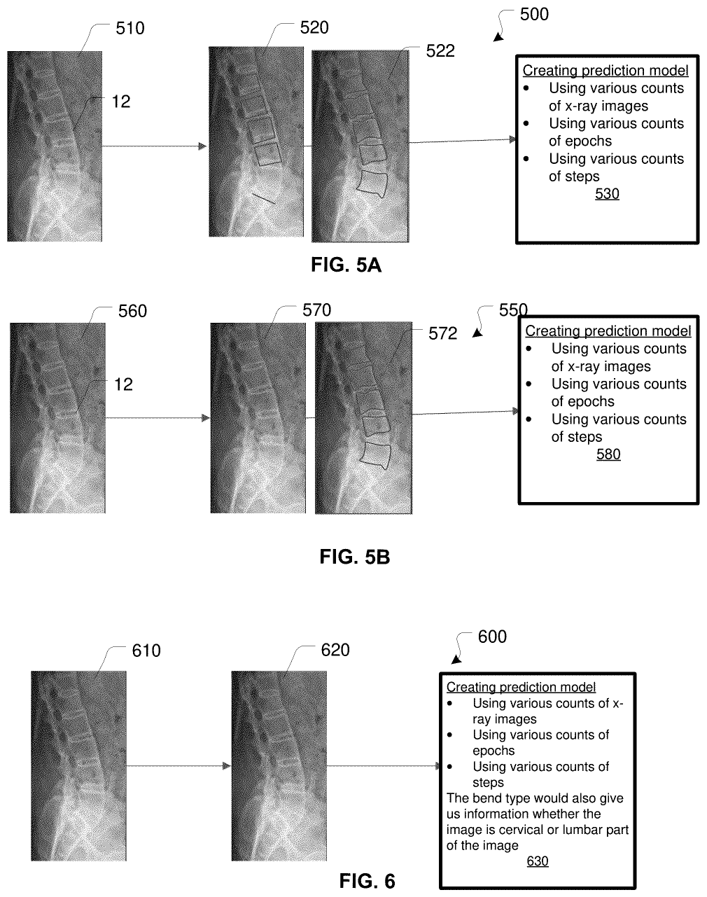

Turning now to FIGS. 5A-B and FIG. 6, training processes are shown. In FIG. 5A, an image, such as an x-ray image, is obtained 510 with a plurality of vertebral bodies 12. A first image 520 with a plurality of points and a second image 522 of the image which is masked are used to extract data for training. A use Mask R-CNN or Detectron2 is used 530 to create a prediction model. The prediction model is created using the vertebral body data organized into a single data set as described in FIG. 4. The data is cleaned to avoid generating a misleading model. Additional variables are applied to evaluate the data.

As would be appreciated by those skilled in the art, Mask R-CNN can be implemented on Python 3, Keras, and TensorFlow. The Mask R-CNN model generates bounding boxes and segmentation masks for each instance of an object in the image. The model is based on Feature Pyramid Network (FPN) and a ResNet101 backbone. Similarly, persons of skill in the art would be familiar with Detectron2 which is Facebook AI Research's next generation library that provides state-of-the-art detection and segmentation algorithms. Detectron2 is the successor of Detectron and maskRCNN-benchmark.

The prediction model can use various counts of images, various counts of epochs (e.g., dates and times from which the computer measures system time) and/or various counts of steps. The various count of steps is a parameter that represents the number of iterations used to run the algorithm and build a model for an epoch. The step count is dynamic and varies based on the specific model being constructed. It can change depending on the complexity and size of the model under consideration. Similarly, the number of epochs is also dynamic, indicating how many times the algorithm iterates over the entire dataset during the training process. Building a model involves running multiple steps for each epoch, making it a combination of various epochs and their corresponding step counts.

Initially a determination is made on how many images to use. Data can then be split between training data and validation data. For example, a mix of 80% training data to 20% validation data, or a mix of 90% training data to 10% validation data. Other combinations can be used without departing from the scope of the disclosure. Training can be started using two classes of data, a generic four point vertebra is V4 and a two point vertebra, S1 or C2, is V2. The two classes are used to create a prediction model with the same number of classes. Masked vertebral data, which could be the COCO dataset format, can be used or the four point (V4) or two point (V2) marked-up data can be used. Training can then be performed on the data using Mask R-CNN or Detectron2. The training can be provide results based on the number of images used, number of epochs, and/or number of steps.

Turning to FIG. 5B, an image, such as an x-ray image, is obtained 560 with a plurality of vertebral bodies 12. A first image 570 with a plurality of points and a second image 572 of the image which is masked are used to extract data for training. A use Mask R-CNN or Detectron2 is used 580 to create a prediction model. Again, the prediction model can use various counts of images, various counts of epochs and/or various counts of steps. Initially a determination is made on how many images to use. Data can then be split between training data and validation data. For example, a mix of 80% training data to 20% validation data, or a mix of 90% training data to 10% validation data. Other combinations can be used without departing from the scope of the disclosure. Validation is used to calculate how accurate the prediction model is. Training can be started using, for example, 5 lumbar vertebra, 12 thoracic vertebra, 8 cervical vertebra and 1 sacral vertebra which is a total of 26 classes. Two of these vertebra (S1, C2) are two point coordinate vertebral bodies and the remaining vertebral bodies have four point coordinates. In some configurations, masked vertebral data can be used, such as a COCO dataset format, or a four point (V4) or two point (V2) marked-up dataset which is marked up by the trained image analysts. Training can then be performed on the data using Mask R-CNN or Detectron2. The training can be provide results based on the number of images used, number of epochs, and/or number of steps.

FIG. 6 shows bend-type recognition training 600. An image with a vertebra is obtained 610. Extracted data is used for training 620. The model is created 630. Bending types are selected from: Calibration Image, Standing Extension, Femoral Heads, Standing Flexion, Lateral Neutral, XTL Supine, XTL Prone, AP Neutral, AP Left, AP Right, Cervical Extension, Cervical Flexion, Cervical Lateral Neutral, Standing Extension, Standing Flexion, Lying Flexion, Lying Extension, Lying Left, Lying Right, Standing Right, Standing Left, Cervical Extension, Cervical Flexion, Archive, Calibration Grid, XR Uncategorized, XR Lateral, XR AP, Lateral AP Neutral, and Calibration Grid. The training can be provide results based on the number of images used, number of epochs, and/or number of steps. Pre-extracted data models are used depending on the training goals for the training model. Preliminarily a decision is made on how many images to use. Data is split, as discussed above, between training and validation. Validation calculates how accurate the prediction model is. Training is started by using bend types associated with the images. Any bend type that is not recognized will be labeled as archived and not used in the diagnosis process. Training is performed using CNN.

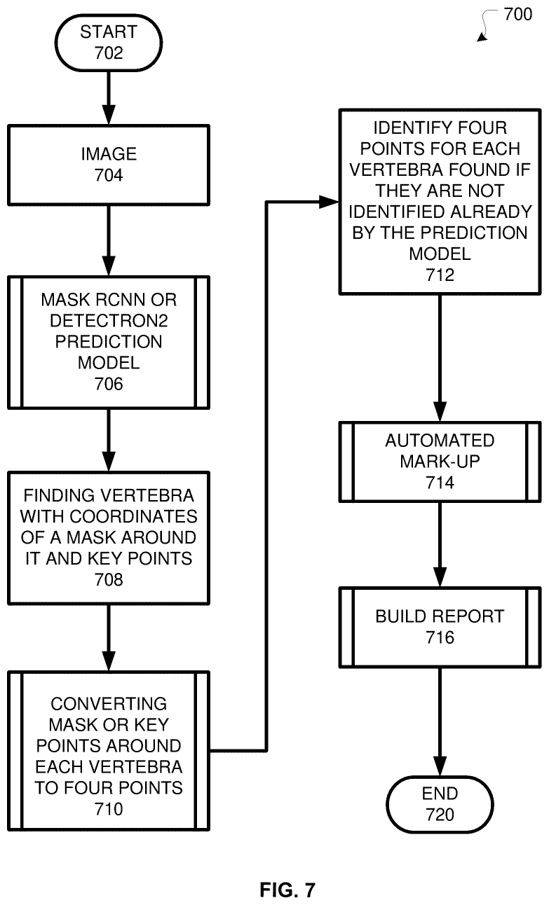

Turning to FIG. 7, a prediction process 700 is shown. The prediction process 700 starts 702. An image is provided 704. The MaskRCNN or Detectron2 prediction model is applied 706. One or more vertebral bodies with coordinates of a mask around it and key points are located 708. The mask or key points are converted around each vertebral body to four points 710. The four points (V4) are identified for each vertebra if not already identified by the prediction model 712. Automated markup 714 are built and reports 716 are generated, after which the process ends 720. By finding all the vertebral bodies on an image using either four point (V4) or two point (V2) marking and using a programmatic approach to identify data, the can be pre-populated on the image and a trained spine image analyst can verify the correctness of the mark-up.

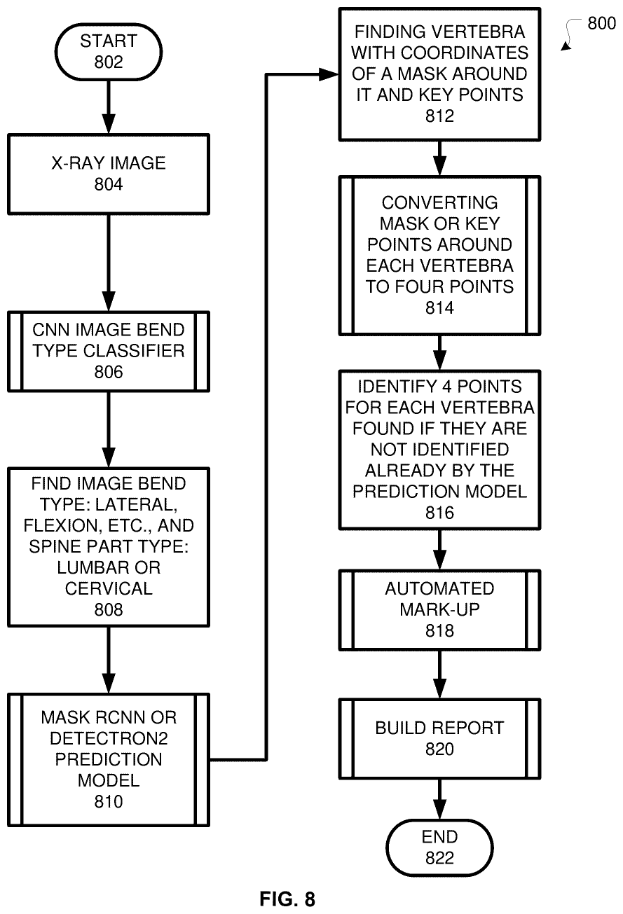

FIG. 8 illustrates another prediction process 800. The prediction process 800 starts 802. An image is provided 804. CNN image bend type classifier 806 is applied. The image bend type is identified 808. Mask R-CNN or Detectron2 prediction model for the specific image type is applied 810. Vertebra with coordinates of a mask around it and key points are found 812. The mask or key points around each vertebra is converted 814. Four points for each vertebra is identified 816. Automated markup 818 and reports 820 are built, after which the process ends 822. Preliminarily, a decision is made on which image bend type is used by the CNN image bend type classifier. A determination is also made about whether to use lumbar or cervical vertebra. A specific prediction is run for each type of image bend model. Alternatively, a generic model can be used instead of a specific model for each bend type. From the prediction model, vertebra information is extracted. All the vertebra on an image are located whether four point (V4) or two point (V2) vertebra. After identifying the vertebra, data is populated on the image and a trained spine image analyst verifies the correctness of the mark-up.

The programs described herein are identified based upon the application for which they are implemented in a specific embodiment of the disclosure. However, it should be appreciated that any particular program nomenclature herein is used merely for convenience, and thus the disclosure should not be limited to use solely in any specific application identified and/or implied by such nomenclature.

A database, such as a first database, can be provided that stores one or more attributes of the system. When a server, such as a first server, is an internet website, the server may be comprised of at least one or more servers and cooperating databases. The platform enables information to be conveniently and efficiently stored from any number of locations. One or more modules may be configured to present an interface to support the intake and output of information for one or more of the functions described herein. The client application may have code scripted to present one or more user interface templates that may be user customizable, have one or more prompted input fields, and/or is configured to work with a browser and a remote server. The server applet works with a browser application resident on the client device and serves one or more web pages to the client device with the resident browser. Communication with remote devices, servers, computers, users, mobile devices, databases, etc. may be in real time or may be at periodic intervals as dictated by the needs and associated functions of the communicated information.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. Flowchart and block diagrams in the Figures illustrate the architecture, functionality, and operation of possible implementations of systems, methods, and computer program products according to various embodiments of the present disclosure. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A backend server can be provided that is further operable to aggregate the received information. Information is then passed to one or more databases and/or one or more users. The one or more databases may receive, store, and disseminate information, The server may be used to communicate and update information stored in the database and communicate to or with one or more associated users in response to the received information. Thus, a software program resident on the server is coded to take in the details, assess the information received, and perform specific functions in response to the received information. The server may then supply information back to each client device to be displayed on a display screen of that client device as well as supply information back to one or more other networked users. The web application on the server can cooperate over a wide area network, such as the Internet or a cable network, with two or more client machines each having resident applications.

The software used to facilitate the protocol and algorithms associated with the disclosed processes can be embodied onto non-transitory machine-readable medium. A machine-readable medium includes any mechanism that provides (e.g., stores and/or transmits) information in a form readable by a machine (e.g., a computer). For example, a machine-readable medium includes read only memory (ROM); random access memory (RAM); magnetic disk storage media; optical storage media; flash memory devices; DVD's, EPROMs, EEPROMs, FLASH, magnetic or optical cards, or any type of media suitable for storing electronic instructions. The information representing the apparatuses and/or methods stored on the machine-readable medium may be used in the process of creating the apparatuses and/or methods described herein. Any portion of the server implemented in software and any software implemented on the client device are both stored on their own computer readable medium in a non-transitory executable format. Embodiments described herein, such as modules, applications, or other functions may be configured as hardware, software, or a combination thereof. The configuration may be stored one a single dedicated device such as an application locally resident and executed on client devices configured to communicate over a network or across many devices such as a website hosted across one or more servers retrieving information across one or more databases, to communicate across a network to a local device, such as laptop, or any combination thereof. Embodiments may also take advantage of cloud computing, such that the exemplary modules, applications, or other functions are stored remotely on one or more servers or devices, and accessed over a network such as the internet or other network connection from an electronic device, such as a mobile device.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, the use of comprise, or variants such as comprises or comprising, includes a stated integer or group of integers but not the exclusion of any other integer or group of integers. It should be understood that various alternatives to the embodiments of the invention described herein may be employed in practicing the invention. It is intended that any claims presented define the scope of the invention and that methods and structures within the scope of these claims and their equivalents be covered thereby.

What is claimed:

1. A vertebral recognition system comprising:
a computer including a memory, a processor and a display; and
an application stored in the memory and executable by the processor of the computer to
obtain a plurality of images from one or more databases wherein the plurality of images include a spine having one or more vertebral bodies,
map one or more vertebral bodies in the one or more images with at least one of two points and four points to generated one or more mapped images of one or more of two point vertebra and four point vertebra,
create a prediction model using the one or more images, wherein the prediction model is created using the plurality of mapped images, and one or more of a count of epochs and various counts of steps, and
build an automated mark-up.

2. The vertebral recognition system of claim 1 wherein the prediction model identifies a bend classification for one or more of the mapped images.

3. The vertebral recognition system of claim 2 wherein the prediction model is selected based on a type of the image bend classification.

4. The vertebral recognition system of claim 1 wherein a determination is made for a number of images to use.

5. The vertebral recognition system of claim 1 further comprising an instruction to split the mapped images into a training category and a validation category.

6. The vertebral recognition system of claim 5 further comprising selecting a percentage of images in the training category and a percentage of images in the validation category.

7. The vertebral recognition system of claim 1 further comprising both two point and four point mapped images.

8. The vertebral recognition system of claim 1 further comprising generate one or more reports.

9. A vertebral recognition system comprising:
a computer including a memory, a processor and a display; and
an application stored in the memory and executable by the processor of the computer to
obtain a plurality of images from one or more databases wherein the plurality of images include a spine having one or more vertebral bodies,
map one or more vertebral bodies in the one or more images with at least one of two points and four points to generated one or more mapped images of one or more two point vertebra and four point vertebra,
create a prediction model using the one or more images, wherein the prediction model is created using the plurality of mapped images, and one or more of a count of epochs and various counts of steps,
instruct to split the mapped images into a training category and a validation category, and
build an automated mark-up.

10. The vertebral recognition system of claim 9 wherein the prediction model identifies a bend classification for one or more of the mapped images.

11. The vertebral recognition system of claim 10 wherein the prediction model is selected based on a type of the image bend classification.

12. The vertebral recognition system of claim 9 wherein a determination is made for a number of images to use.

13. The vertebral recognition system of claim 9 further comprising selecting a percentage of images in the training category and a percentage of images in the validation category.

14. The vertebral recognition system of claim 9 further comprising both two point and four point mapped images.

15. The vertebral recognition system of claim 9 further comprising generate one or more reports.

16. A method of performing a surgical procedure, comprising:
storing a software application on a memory associated with a computer, which when executed by a processor, causes the processor to
obtain a plurality of images from one or more databases wherein the plurality of images include a spine having one or more vertebral bodies,
map one or more vertebral bodies in the one or more images with at least one of two points and four points to generated one or more mapped images of one or more of two point vertebra and four point vertebra,
create a prediction model using the one or more images, wherein the prediction model is created using the plurality of mapped images, and one or more of a count of epochs and various counts of steps,
build an automated mark-up,
generate a surgical procedure plan based on the prediction model.
